(19) United States
(12) Patent Application Publication
(10) Pub. No.: US 2025/0381220 A1
(43) Pub. Date: Dec. 18, 2025

(54) MAGNESIUM-L-THREONATE AND NEUROTRANSMITTER COMPOSITIONS AND RELATED METHODS

(71) Applicant: Neuro Supplements, LLC, City of Industry, CA (US)

(72) Inventors: Jennifer Gu, Diamond Bar, CA (US); Guosong Liu, Oakland, CA (US)

(73) Assignee: Neuro Supplements, LLC, City of Industry, CA (US)

(21) Appl. No.: 19/199,690

(22) Filed: May 6, 2025

Related U.S. Application Data
(63) Continuation of application No. 17/194,733, filed on Mar. 8, 2021, now abandoned.
(60) Provisional application No. 62/986,375, filed on Mar. 6, 2020.

(51) Int. Cl. A61K 33/06; A61K 31/191; A61K 31/685; A61K 45/06

(52) U.S. Cl. CPC A61K 33/06; A61K 31/191; A61K 31/685; A61K 45/06

(57) ABSTRACT

The present composition comprises magnesium-L-threonate and a neurotransmitter such as, but not limited to phosphatidylserine and optionally, at least one of vitamin C, vitamin B6, and vitamin D3. The composition has been shown to increase at least one of memory or cognition in clinical study subjects. The present disclosure is further directed to methods of identifying and a subject with a memory or cognitive deficiency and administering a therapeutically effective amount of a formula comprising magnesium-L-threonate and a neurotransmitter to improve the memory and/or cognitive deficiency. In some implementations, the magnesium-L-threonate is Magtein®. It is also intended that any other neurotransmitter that results in a synergistic composition of the magnesium-L-threonate and the neurotransmitter by administered to improve a subject's memory and/or cognition.

### CROSS REFERENCE TO RELATED APPLICATIONS

This application is a continuation of U.S. patent application Ser. No. 17/194,733 filed on Mar. 8, 2021, entitled “Magnesium-L-Threonate and Neurotransmitter Compositions and Related Methods” which claims priority to U.S. Provisional Patent Application No. 62/986,375 filed on Mar. 6, 2020, entitled “Magnesium-L-Threonate and Neurotransmitter Compositions, and Related Methods” to Gu, et al., the contents of which are hereby incorporated by reference in their entirety.

### BACKGROUND

### 1. Field of the Invention

Exemplary embodiments generally relate to compositions comprising magnesium-L-threonate and a neurotransmitter such as phosphatidylserine and citicoline, and optionally for instance vitamins and methods of using the compositions to improve memory and cognition.

### 2. Related Art

Magnesium is the second most abundant intracellular minerals and it is required as a cofactor for over 300 enzymatic reactions and is thus necessary for the biochemical functioning of numerous metabolic pathways in our body, including energy generation in every cell, protein production, gene regulation, bone and teeth maintenance, as well as the proper function of the brain and nervous systems (SWAMINATHAN, 2003, JAHNEN-DECHENT; KETTELER, 2012, ZARATE et al., 2013, SCHWALFENBERG; GENUIS, 2017, SCHWALFENBERG; GENUIS, 2017). It is known that magnesium is important for almost all cognitive functions. However, most magnesium compounds available on the market have low bioavailability in the cognitive tissue because they cannot cross the blood-brain barrier easily and have severe adverse effects on the gastrointestinal tract (Fuchs-Buder et al., 1997; McKee et al., 2005). Thus, it is of great interest for cognitive research community to identify a brain bioavailable Magnesium source.

In a Neuron publication of 2010, scientists from the Massachusetts Institute of Technology (MIT) discovered a magnesium compound called magnesium-L-threonate (Magtein®), that can effectively deliver magnesium to brain cells (SLUTSKY et al., 2010). According to the study, magnesium-L-threonate (Magtein®) is a superior magnesium source to other magnesium compounds because it has a higher bioavailability when compared to other sources of magnesium, such as chloride, citrate, glycinate and gluconate, demonstrated by higher absorption and higher retention. In addition, it was able to significantly elevate magnesium concentrations (7% to 15% of initial value in 24 days) in cerebrospinal fluid in rats when administered orally, while other magnesium compounds could not. In humans, L-threonine acid is an ascorbic acid metabolite (FAY; VERLANGIERI, 1991), having been identified in plasma (DEUTSCH et al., 1999), in the aqueous humor (HARDING et al., 1999), in the urine (THOMPSON et al., 1975; LAWSON et al., 1976) and in the brain (Sun et al., 2016). In addition to its endogenous occurrence, L-threonine acid can be found in a wide variety of foods, such as canned mushrooms, fruit juice and processed meats (AZZAM et al., 2011), as the major part of Vitamin C metabolites.

Slutsky and colleagues reported that after one month of magnesium L-threonate (Magtein®) supplementation, the concentration of magnesium in the brain increased, and there was a significant improvement in memory and learning in both young rats and in elderly rats. In addition, magnesium-L-threonate improved memory recovery in elderly rats. Magnesium-L-threonate supplementation did not influence body weight, motility and amount of water and food intake. The possible mechanisms of action of magnesium L-threonate, on cognitive functions were also demonstrated to function via the activation of the NMDA receptors, which leads to increased synaptic density and improved memory (SLUTSKY et al., 2010).

Based on these preclinical results, it would be of great interest to investigate the effect of magnesium-L-threonate in human population since cognitive impairment is a major problem in elderly. A randomized, double-blind, placebo-controlled study in older American adults (between age 50-70) were published in 2016 (Liu et al., 2016), where supplementation with magnesium-L-threonate significantly improved overall cognitive scores as compared to placebo (p=0.003; Cohen's d=0.91). Cognitive fluctuation was also reduced. The study population had more severe executive function deficits than age matched controls and magnesium-L-threonate treatment nearly restored their impaired executive function, demonstrating that magnesium-L-threonate may be clinically significant.

### SUMMARY

The present invention is directed to a composition comprising magnesium-L-threonate and a neurotransmitter. In some embodiments, the neurotransmitter may be phosphatidylserine which may be derived from one of or any combination of soy, sunflower, fish, krill, and bovine. In some embodiments, the magnesium-L-threonate is Magtein®. In some embodiments, the neurotransmitter may be at least one of phosphatidylserine, citicoline, and gamma-aminobutyric acid. In some embodiments, the neurotransmitter is at least one of acetylcholine, dopamine, gamma-aminobutyric acid, glutamate, histamine, norepinephrine, and serotonin. In some embodiments, the neurotransmitter is at least one of an amino acid, a gasotransmitter, a monoamine, a trace amine, a peptide, a purine, and a catecholamine. In some embodiments, the composition may comprise at least one of vitamin C, vitamin D3, vitamin B6, and vitamin B12.

The present invention is also directed to a method of improving at least one of memory and cognition in a subject by identifying a subject having a deficiency in at least one of memory and cognition and administering a therapeutically effective amount of a composition comprising magnesium-L-threonate and a neurotransmitter. In some embodiments, the neurotransmitter may be phosphatidylserine which may be derived from one of or any combination of soy, sunflower, fish, krill, and bovine. In some embodiments, the magnesium-L-threonate is Magtein®. In some embodiments, the neurotransmitter may be at least one of phosphatidylserine, citicoline, and gamma-aminobutyric acid. In some embodiments, the neurotransmitter is at least one of acetylcholine, dopamine, gamma-aminobutyric acid, glutamate, histamine, norepinephrine, and serotonin. In some embodiments, the neurotransmitter is at least one of an amino acid, a gasotransmitter, a monoamine, a trace amine, a peptide, a purine, and a catecholamine. In some embodiments, the composition may comprise at least one of vitamin C, vitamin D3, vitamin B6 and vitamin B12.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventors' intent and desire that the simple, plain and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112 (f). Thus, the use of the words “function,” “means” or “step” in the Detailed Description or Brief Description of the Drawings or claims is not intended to somehow indicate a desire to invoke the special provisions of 35 U.S.C. § 112 (f), to define the invention. To the contrary, if the provisions of 35 U.S.C. § 112 (f) are sought to be invoked to define the inventions, the claims will specifically and expressly state the exact phrases “means for” or “step for, and will also recite the word “function” (i.e., will state “means for performing the function of [insert function]”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventors not to invoke the provisions of 35 U.S.C. § 112 (f). Moreover, even if the provisions of 35 U.S.C. § 112 (f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

The foregoing and other aspects, features, and advantages will be apparent to those artisans of ordinary skill in the art from the DETAILED DESCRIPTION and DRAWINGS, and from the CLAIMS.

### BRIEF DESCRIPTION OF THE DRAWINGS

Example embodiments will become more fully understood from the detailed description given herein below and the accompanying drawings, which are given by way of illustration only and thus are not limitative of the example embodiments herein. Elements and acts in the drawings are illustrated for simplicity and have not necessarily been rendered according to any particular sequence or embodiment.

FIG. 1 is a chart showing a comparison of the composite score of overall brain function of Magtein® versus placebo.

FIG. 2 is a chart showing the effects of a formular comprising Magtein® and phosphatidylserine (PS) on cognitive functioning versus placebo.

FIG. 3 is a chart showing a comparison of cognitive improvement of Magtein® versus Magtein® and PS.

FIG. 4 is a chart showing time spent in target quadrants in Morrris Water Maze (MWM) tests in animals.

### DETAILED DESCRIPTION

Magnesium is one of the most abundant minerals in the body. It has an essential physiological role. However, most magnesium compounds have low bioavailability in cognitive tissues because they cannot cross the blood-brain barrier easily. In 2010, MIT scientists identified a magnesium compound, Magnesium-L-threonate, that was able to cross the blood brain barrier and raise magnesium levels in the brain effectively. Magnesium-L-threonate is commercially available as Magtein®.

The below definitions and discussion are intended to guide understanding but are not intended to be limiting with regard to other disclosures in this application. References to percentage (%) in compositions disclosed herein refer to the % by weight of a given component to the total weight of the composition, also signified by “w/w”, unless stated otherwise.

In the present disclosure, a composition comprises magnesium-L-threonate and a neurotransmitter. In some embodiments, the magnesium-L-threonate is provided in powdered form. In some embodiments, the magnesium-L-threonate is Magtein®. In some embodiments, the neurotransmitter comprises at least one of phosphatidylserine, citicoline, citicoline sodium, and gamma-aminobutyric acid (GABA). In some embodiments, the neurotransmitter comprises at least one of the following categories of neurotransmitters: acetylcholine, dopamine, GABA, glutamate, histamine, norepinephrine, and serotonin. In some embodiments, the neurotransmitter comprises at least one of the following major categories of neurotransmitters: amino acids, gasotransmitters, monoamines, trace amines, peptides, purines, catecholamines, and others such as by non-limiting example, acetylcholine (Ach) and anandamide. Amino acids may comprise, by non-limiting example, glutamate, aspartate, D-serine, GABA, and glycine. Gasotransmitters may comprise, by non-limiting example, nitric oxide (NO), carbon monoxide (CO), and hydrogen sulfide (H₂S). Monamines may comprise, by non-limiting example, dopamine (DA), norepinephrine (noradrenaline, NE, NA), epinephrine (adrenaline), histamine, and serotonin (SER, 5-HT). Trace amines may comprise, by non-limiting example, phenethylamine, N-methylphenethylamine, tyramine, 3-iodothyronamine, octopamine, and tryptamine. Peptides may comprise, by non-limiting example, oxytocin, somatostatin, substance P, cocaine and amphetamine regulated transcript, and opioid peptides. Purines may comprise, by non-limiting example, adenosine triphosphate (ATP) and adenosine. Catecholamines may comprise, by non-limiting example, dopamine, norepinephrine (noradrenaline), and epinephrine (adrenaline). In Examples 2 and 3 described below, the neurotransmitter is phosphatidylserine, however, any of the other neurotransmitters and neurotransmitter categories described above are believed to have increased synthesis, decreased degradation, or enhanced releasing and activities of these molecules when combined with magnesium-L-threonate. Any of the neurotransmitters listed above may be produced using conventional manufacturing practices to derive the neurotransmitter into supplement form. By non-limiting example, when phosphatidylserine (PS) is derived from soy, the phosphatidylcholine-enriched lecithin is enzymatically trans-phosphatidylated with L-serine using phospholipase D, which catalyzes the substitution of the choline head-group with serine to form PS. Following the enzymatic reaction, PS is separated from the reaction mixture, purified, and dried. In some embodiments, the composition is a synergistic composition that provides effects such as improvement of memory or cognition that is statistically significant from Magtein® or other magnesium-L-threonate alone or from the neurotransmitter(s) alone.

A composition may comprise, consist essentially of, or consist of, magnesium-L-threonate and a neurotransmitter. In some embodiments, a combination of magnesium-L-threonate and a neurotransmitter, when administered together to the body, improve memory and cognition in a subject. Magtein® according to the present disclosure is magnesium-L-threonate in the form of a dietary supplement, available under the name Magtein®. In some embodiments, Magtein® is magnesium-L-threonate monohydrate. In some embodiments, Magtein® is a white to off-white powder and is water soluble, forming a clear solution when the powder is dissolved in water, 1% powder at 25° C. In some embodiments, bulk density is not less than about 0.4 g/cc (grams per cubic centimeter), particle size is not less than 90% through a US #20 and not more than 60% through a US #20, and/or loss on drying is not more than 5%. In some embodiments, Magtein® includes about 7.2% to about 8.3% magnesium (by ICP-MS), not less than about 85% L-threonic acid (by HPLC) and/or not more than about 5000 ppm residual solvents (ethanol) (by GC-MS). In some embodiments, Magtein® includes about 86% to about 91% L-threonic acid. In some embodiments, Magtein® in aqueous solution (1% water) has a pH of 5.8-7.0, arsenic in an amount not more than about 0.5 ppm and/or cadmium in an amount of not more than about 0.2 ppm, lead in an amount of not more than about 0.5 ppm, and/or cadmium in an amount of not more than about 0.2 ppm (all except pH by ICP-MS). In some embodiments, total microbiological plate count of Magtein® is not more than about 3000 cfu/g, yeast and mold are not more than about 300 cfu/g and/or Magtein® is negative for E. coli and Salmonella (all by USP 2021 except Salmonella, by AOAC 998.09). In some embodiments, Magtein® does not contain animal sourced raw materials and is suitable for vegetarians and vegans. In some embodiments, Magtein® is free of genetically modified organisms (GMO) and bovine spongiform encephalopathy/transmissible spongiform encephalopathy (BSE/TSE) risk. In some embodiments, Magtein® is non-irradiated and non-ETO treated. In some embodiments, the shelf life of Magtein® is three years from the date of manufacture. In some embodiments, any or all of the above embodiments of Magtein® apply. In some embodiments, a CAS # is 500304-46-7.

In some embodiments of the present disclosure, a composition further comprises a vitamin such as one or more of the following: alpha-glycerophosphocholine (Alpha GPC); Alpha Lipoic Acid; CoEnzymeQ10; Dimethylaminoethanol Bitartrate (DMAE Bitartrate); Thiamine HCL (Vitamin B1); Vitamin B12 USP; D-Calcium Pantothenate (Vitamin B5); Vitamin D3; Bacopa; and Ocibest. In some embodiments, the composition includes one or more of Vitamin D3, Vitamin C, and Vitamin B6. The vitamins may be provided, by non-limiting example, in daily amounts of about 40 to about 5000 IU Vitamin D3 about 25 to about 200 mg Vitamin C, and about 10 to about 50 mg Vitamin B6. The vitamins may be provided, by non-limiting example, in daily amounts of about 200 to about 400 IU Vitamin D3, about 40 to about 60 mg Vitamin C, and about 15 to about 20 mg Vitamin B6. In some embodiments, a daily dose of the composition is administered in part in the morning and in part in the evening before sleep.

A composition of the present disclosure may be, by non-limiting example, in powdered from or in liquid form, or any other acceptable forms for administration. A composition may further comprise one or more excipients, additives, and/or other substances, including, by non-limiting example, microcrystalline cellulose, cellulose, rice flour, magnesium stearate, and silica. A composition may be formulated into nutraceutical or pharmaceutical dosage forms comprising, by non-limiting example, tablets, capsules, powders, liquids, chews, gummies, transdermals, injectables, dietary supplements, topical creams or gels, lozenges, pills, and so forth. In some embodiments, a composition of the present disclosure is described in one or more of the Examples below.

In some embodiments, a composition of the present disclosure is administered in an effective amount to a mammal, including a daily dose for a human being of about 100 to about 3000 mg magnesium-L-threonate per day with a neurotransmitter. In some embodiments, the neurotransmitter may comprise PS with a daily dosage of about 50 mg to about 500 mg. By non-limiting example, the daily dose of magnesium-L-threonate may be about 1000 to about 2000 mg per day or may be about 1600 mg per day. Also by non-limiting example, the daily dose of PS may be about 100 mg to about 300 mg or about 200 mg per day. In some embodiments, the neurotransmitter is phosphatidylserine. In a different mammal, such as by non-limiting example, a horse, dog, or cat, a dose may be proportionally same or similar to that of a human, adjusted per kilogram of weight of the animal.

By non-limiting example, a composition of the present disclosure may be provided in a capsule having about 400 mg magnesium-L-threonate (Magtein®, as shown in Example 2, below), about 50 mg phosphaditylserine, about 80 IU Vitamin D3, about 12 mg Vitamin C, and about 4 mg Vitamin B6. After administration of four capsules to a human subject (two capsules in the morning and two at night before sleep) for 30 days, improvements in memory and/or cognition were shown by test results as discussed in Example 2.

A “dietary supplement” according to the present disclosure refers to a composition that may be orally administered as an addition to a subject's diet, which is not a natural or conventional food, which when administered improves memory and/or cognition in the subject, as discussed throughout this application. In some embodiments, the dietary supplement is administered according to any of the following dosing schedules: daily for at least one week, one day to one week, two weeks, three weeks, four weeks, 30 days, five weeks, 6 weeks, 7 weeks, 8 weeks, up to 26 weeks, or chronically for at least one day to three months, six months, nine months, or one year or more, or for another period of time according to the present disclosure.

In the present disclosure, “administering,” “administration,” and the like refer to providing a composition to a subject in an effective amount, that is, an amount effective for the magnesium-L-threonate and the neurotransmitter to reach the subject's bloodstream and/or tissues, and depending on the route of administration, pass the blood-brain barrier, ultimately reaching cells for improving memory and/or cognition, and acting on those cells to improve cognition and/or memory, for instance, as in Examples 2 and 3 below. Administration may be made by the subject or another. Administration to the subject maty be oral, by non-limiting example, in the form of a dietary supplement, and/or in a solid dosage form, such as by non-limiting example, in a discrete dose unit. Administration may also be through parenteral, intramuscular, transdermal, topical, sublingual, intravenous, and other physiologically acceptable routes. A subject of the disclosure herein may be a human or another animal such as by non-limiting example, a dog, cat, horse, or other mammal.

According to the present disclosure, “improving memory” and/or “improving cognition” refers to, by non-limiting example, a test result showing memory or cognition is improved after administration of a composition, compared with before administration. In a synergistic composition as disclosed herein, the improved test result shows a statistically significant improvement of memory and/or cognition after administration of a composition containing magnesium-L-threonate and a neurotransmitter as compared with magnesium-L-threonate alone or the neurotransmitter alone. In some embodiments, the improved cognition and/or memory may be determined without a formal test.

According to the present disclosure, a subject having a memory and/or cognition impairment or deficiency, for example, from a diagnosed condition or due to a personal perception of impairment or deficiency, may be treated with a composition as disclosed to improve memory and/or cognition.

Recitations of ranges of values herein are merely intended to serve as a shorthand method of referring individually to separate values falling within the range, unless otherwise indicated herein, and each separate value is incorporated into the specification as if it were individually recited herein. Use of the term “about” is intended to describe values either above or below the stated value in a range of approximately ±20% in some embodiments. In other embodiments, the values may range in value above or below the stated value in a range of approximately ±5%. In other embodiments, the values may range in value above or below the stated valued in a range of approximately ±2%. In other embodiments, the values may range in value above or below the stated value in a range of approximately ±1%. The proceeding ranges are intended to be made clear by context and no further limitation is implied. All method steps described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise stated. No language in the specification should be construed as indicating any non-claimed element as essential to the practice of the invention.

The present invention may be further understood in connection with the following Examples and embodiments. The following non-limiting Examples and embodiments described herein are provided to illustrate the invention and are not intended to be construed as limiting embodiments.

In Example 1, below, a magnesium-L-threonate formulation was subjected to a human clinical study where a randomized, double-blind, placebo-controlled, parallel-designed trial in older adult subjects (age 50-70) with cognitive impairment were recruited.

### Example 1

Subjects were given with MMFS-01 (Magnesium-L-Threonate+Vitamin C+Vitamin D) (n=23) or placebo (n=21) for 12 weeks and cognitive ability, sleep quality, and emotion were evaluated. MMFS-01 contains the following: 900 mg of Magtein® X2, 30 mg of ascorbic acid X2, and 200 IU of Vitamin D3 X2. The small amounts of Vitamins C and D were included in the MMFS-01 to prevent variations due to extreme Vitamin C or D deficiency in subjects. At these dosages, Vitamins C and D are not expected to be efficacious on cognitive function of the brain.

Scores from several cognitive tests, evaluating four domains of cognition—executive function, working memory, attention, and episodic memory—were combined to produce a composite score (Z) to assess overall cognitive ability. See Liu, G., Weinger, J. G., Lu, Z. L., Xue, F. Sadeghpour, S. (2016) Efficacy and Safety of MMFS-01, a Synapse Density Enhancer, for Treating Cognitive Impairment in Older Adults: A Randomized, Double-Blind Placebo-Controlled Trial J. Alzheimers Dis. 49 (4): 971-990, the content of which is hereby incorporated by reference in its entirety.

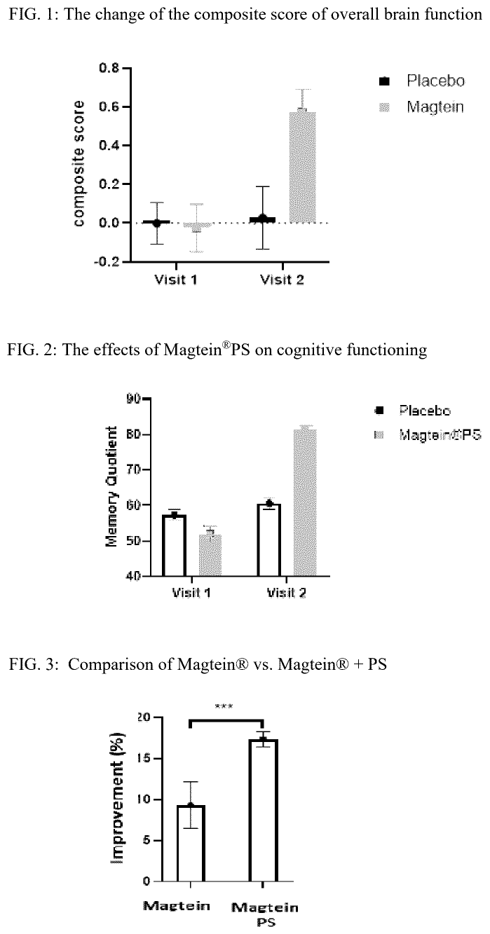

As shown in FIG. 1, the difference of change of the composite score from visit 2 (12 weeks) to visit 1 (baseline) between MMFS-01 vs placebo is 0.633 SD (p<0.001). The possible range of composite scores is between-3 to 3, therefore, the percentage of improvement in MMFS-01 vs placebo is 10.5%.

Thus, it can be concluded that the MMFS-01 formula results in a statistically significant improvement in cognitive ability, sleep quality, and emotion among human subjects.

In Example 2, below, a Magtein®-based formulation that includes the neurotransmitter phosphatidylserine was tested in a Chinese human population between the ages of 18-65 years. “The clinical memory test,” the standard test commonly used in Chinese Hospitals and Academic Institutes for cognitive evaluation was applied to 109 healthy volunteers. The test consists of 5 subtests: directed memory, paired-association learning, free recall of pictures, recognition of meaningless figures, recall of the connection between portraits and their characteristics (Lin, 2003). After 30 days, memory quotient of the experimental group was significantly higher than the control group, showing significant benefits of this Magnesium-L-threonate and phosphatidylserine-based formula in improving memory and cognition in humans.

### Example 2

### A. Methodology

### 1. Materials

Magnesium-L-threonate (Magtein®) based formula and placebo: The following Magtein®-based formula for Experimental group (each capsule):

| | | |
|---|---|---|
| Magnesium-L-threonate (Magtein®): | 400 | mg |
| Vitamin D3: | 80 | U |
| Vitamin C: | 12 | mg |
| Vitamin B6: | 4 | mg |
| Phosphatidylserine | 50 | mg |

Specification: 0.54 g/capsule, recommended dose is 2.16 g/person/day. Daily dosage is 2 capsules in the morning and 2 capsules in the evening before sleep.

Control subjects were provided capsules containing microcrystalline cellulose as a placebo.

### 2. Principle of Subject Selection

This clinical trial was conducted in the Center for Health Safety of Nanjing Medical University and its affiliated hospital located in Hefei, Anhui, PRC. Subjects were chosen from 18 to 65 years old with good health condition and were willing to participate by signing the consent form. All voluntary subjects were selected with similar influencing factors, including similar education level; similar age; and without previous experience of similar tests. Subjects who have taken any food, drink, or medication that can influence the study were also excluded.

Comparison analyses were performed in two aspects: self and groups.

Experimental group and control group were randomly selected with age and gender balanced. Results of 102 subjects were collected at the end of trial, 51 for experimental group, and 51 for control group.

### 3. Testing Methods

The commonly used clinical cognition test, “The Clinical Memory Test”, revised by the Institute of Psychology of the Chinese Academy of Science in 1996, was applied to 109 healthy volunteers. “The Clinical Memory Test” is the standard test commonly used in Chinese Hospitals and Academic Institutes for cognitive evaluation. It consists of 5 subtests: directed memory, paired-association learning, free recall of pictures, recognition of meaningless figures, recall of the connection between portraits and their characteristics (Lin, 2003). Comparison analysis were performed in two aspects: self and groups. After 30 days of supplementation, results of 102 subjects were collected and compared.

### 4. Data Analysis

Parallel comparison between groups were analyzed by using t-test of two samples' mean. Self-reference data were analyzed by using paired t-test. When variance is uneven, data conversion was done by using t′-test and rank sum test. Effective rate and total effective rate were calculated via x² test. In the case that the total number of cases in the four-square table is less than 40, or the total number of cases is equal to or greater than 40, but the theoretical number is equal to or less than 1, the exact probability method is used instead.

### 5. Instruments

Automatic biochemical analyzer (A25Biosystems); automatic hematology analyzer (BC-3000 Plus); automatic urine analyzer (Uritest-300); B-ultrasound set (RH-3200); X-ray machine (Imax-1500z); and ECG machine (AIKD-B-12).

### 6. Safety Parameters

The following tests were measured for all participants as safety parameters:
General health indicators: mental condition, sleep, diet, excretion, heart rate, blood pressure;
Blood/urine test: blood test includes white blood cells, red blood cells, hemoglobin and platelets;
Blood biochemical test: checking list includes serum total protein, albumin, alanine, aminotransferase, aspartate aminotransferase, urea, creatinine, cholesterol, triglycerides, blood glucose;
Chest X-rays, ECG, B-ultrasound tests: only performed once at the beginning of the study.

### B. Results

### 1. Background Distribution Between Experimental Group and Control Group

Data of 102 subjects was collected, which includes experimental group with 51 subjects, 24 male and 27 female, average age 41.04; control group with 51 subjects, 19 male and 32 female, average age 42.47. As seen in Table 1, the differences of MQ, age, gender, education level between experimental group and control group are not statistically significant (P>0.05). It indicates that two groups are comparable in MQ, age, gender and education level at the beginning of the study. No abnormality was observed from chest X-rays, ECG or abdominal B-ultrasound tests from both groups.

TABLE 1
Baseline distribution comparison of two groups before clinical trial.

| Index | Experimental group | Control group | P |
|---|---|---|---|
| MQ | 60.31 ± 11.35 | 60.75 ± 12.31 | 0.854 |
| Age (years) | 41.04 ± 9.41 | 42.47 ± 9.40 | 0.444 |
| Male/Female | 24/27 | 19/32 | 0.423 |

Note:
P represents the comparison between Experimental group and Control group.

### 2. General Health Indicators Between Experimental Group and Control Group

All subjects were monitored on mental condition, sleep, diet and regularity throughout the study. Data was collected and categorized in good, normal, and bad as different levels. Blood pressure and heart rate were also tested. During the clinical study period (Table 2), the magnesium-L-threonate and phosphatidylserine-based formula had no negative effect on the parameters evaluated.

TABLE 2
General Comparison between two groups before and after clinical trial.

| | Experimental group | | | | | | | Control group | | | | | | |
|---|---|---|---|---|---|---|---|---|---|---|---|---|---|---|
| | Before clinical trial | | | After clinical trial | | | | Before clinical trial | | | After clinical trial | | | |
| | Good | Normal | Bad | Good | Normal | Bad | P¹ | Good | Normal | Bad | Good | Normal | Bad | P¹ |
| Mental condition | 50 | 1 | 0 | 51 | 0 | 0 | 1.000 | 51 | 0 | 0 | 51 | 0 | 0 | 1.000 |
| Sleep condition | 50 | 1 | 0 | 51 | 0 | 0 | 1.000 | 51 | 0 | 0 | 51 | 0 | 0 | 1.000 |
| Apatite | 50 | 1 | 0 | 51 | 0 | 0 | 1.000 | 51 | 0 | 0 | 51 | 0 | 0 | 1.000 |
| Exercise | 50 | 1 | 0 | 51 | 0 | 0 | 1.000 | 51 | 0 | 0 | 51 | 0 | 0 | 1.000 |

| | | | | | | |
|---|---|---|---|---|---|---|
| Systolic Blood | 120.00 ± 8.60 | 119.31 ± 7.36 | 0.184 | 119.65 ± 7.40 | 119.65 ± 7.40 | 0.124 |
| pressure(mmHg) | | | | | | |
| Diastolic Blood | 76.24 ± 3.94 | 76.25 ± 3.82 | 0.957 | 76.55 ± 3.60 | 76.55 ± 3.60 | 0.203 |
| pressure(mmHg) | | | | | | |
| Heart rate | 69.39 ± 9.69 | 70.37 ± 7.56 | 0.070 | 71.20 ± 9.87 | 70.82 ± 9.87 | 0.583 |

Note:
P¹ represents the self-comparison before and after clinical trial.

### 3. Effects on the Participants' Blood and Urine Biochemical Parameters

As shown in Table 3, the blood biochemical levels of participants in both groups were in normal range before and after the study. Urine tests from both groups were also normal before and after the study. Therefore, the formulation containing magnesium-L-threonate and phosphatidylserine had no negative effects on blood, or urine biochemical parameters of all participants.

TABLE 3
The effect of magnesium-L-threonate and phosphatidylserine
on levels of human blood and urine biochemical parameters.

| | Before clinical trial | | | After clinical trial | | |
|---|---|---|---|---|---|---|
| | Experimental | Control | | Experimental | Control | |
| Index | group | group | P | group | group | P |
| Leukocyte (10⁹/L) | 6.15 ± 1.73 | 5.90 ± 1.17 | 0.389 | 6.25 ± 1.42 | 5.87 ± 1.46 | 0.180 |
| RBC (10¹²/L) | 4.56 ± 0.6 | 4.38 ± 0.44 | 0.084 | 4.62 ± 0.53 | 4.46 ± 0.46 | 0.110 |
| Platelet (10⁹/L) | 176.2 ± 48.65 | 176.2 ± 48.21 | 1.000 | 188.61 ± 50.6 | 200.22 ± 46.34 | 0.230 |
| Hemoglobin (g/L) | 136.27 ± 17.79 | 130.9 ± 17.93 | 0.132 | 135.94 ± 16.57 | 130.59 ± 19.36 | 0.137 |
| Total protein (g/L) | 71.70 ± 3.82 | 71.05 ± 3.09 | 0.352 | 74.54 ± 5.38 | 75.35 ± 3.78 | 0.384 |
| Albumin (U/L) | 46.38 ± 2.46 | 45.39 ± 2.29 | 0.067 | 48.15 ± 4.67 | 48.51 ± 2.53 | 0.627 |
| Alanine Aminotransferase (U/L) | 20.68 ± 15.00 | 23.7 ± 25.11 | 0.464 | 20.1 ± 12.92 | 24.00 ± 27.38 | 0.359 |
| Aspartate transaminase U/L) | 19.39 ± 5.61 | 20.2 ± 12.23 | 0.671 | 19.04 ± 4.97 | 20.88 ± 12.66 | 0.335 |
| Urea (mmol/L) | 5.6 ± 1.6 | 5.31 ± 1.32 | 0.325 | 5.03 ± 1.36 | 4.90 ± 1.38 | 0.624 |
| Creatinine (umol/L) | 62.73 ± 11.16 | 59.61 ± 14.62 | 0.229 | 72.78 ± 13.80 | 68.8 ± 15.9 | 0.180 |
| Blood sugar (mmol/L) | 5.68 ± 0.5 | 6.08 ± 1.68 | 0.101 | 4.72 ± 0.57 | 5.17 ± 1.23 | 0.071 |
| Cholesterol (mmol/L) | 5.37 ± 0.69 | 5.18 ± 0.88 | 0.213 | 4.67 ± 0.85 | 4.53 ± 0.88 | 0.408 |
| Triglyceride (mmol/L) | 1.55 ± 1.83 | 1.31 ± 0.71 | 0.385 | 1.61 ± 1.45 | 1.63 ± 1.85 | 0.953 |
| Urine test | Normal | Normal | | Normal | Normal | |

Note:
P¹ represents the self-comparison before and after clinical trial.

### 4. The Effect of the Magnesium L-Threonate and Phosphatidylserine-Based Formula on Directed Memory Test of Subjects

As seen in Table 4, the average score of directed memory test has no significant difference (P>0.05) between experimental group and control group before the clinical trial. After 30 days, average score of directed memory test in experimental group was significantly higher than control group (P<0.001). In self-comparison, average score of experimental group after the trial was significantly higher than before (P<0.05); while in placebo group, there was no significant difference between the average score of directed memory before and after the trial (P>0.05).

TABLE 4
The effect of magnesium-L-threonate and phosphatidylserine
on scores of directed memory test.

| | | Before | | After | | Increased | | |
|---|---|---|---|---|---|---|---|---|
| Groups | Subjects | Trial | P | Trial | P | Value | P | P¹ |
| Experimental | 51 | 10.69 ± 4.98 | 0.828 | 17.20 ± 4.26 | 0.000 | 6.51 ± 3.47 | 0.000 | 0.000 |
| Control | 51 | 10.47 ± 5.00 | | 10.98 ± 3.94 | | 0.51 ± 2.94 | | 0.222 |

Note:
P represents the comparison between Experimental group and Control group.
P¹ represents the self-comparison before and after clinical trial.

### 5. The Effect of the Magnesium-L-Threonate and Phosphatidylserine-Based Formula on Paired-Association Learning Test

As shown in Table 5, the average score of paired-association learning test has no significant difference (P>0.05) between experimental group and control group before the clinical trial. Average score of paired-association learning test in experimental group was higher than control group after the trial, with statistical significance (P<0.001).

In self-comparison, average score of experimental group after the trial was higher than before, with statistical significance (P<0.001). There is no significant difference of the average score of paired-association learning in placebo group before and after the trial (P>0.05).

TABLE 5
The effect of magnesium-L-threonate and phosphatidylserine
on scores of paired-association learning.

| | | Before | | After | | Increased | | |
|---|---|---|---|---|---|---|---|---|
| Groups | Subjects | Trial | P | Trial | P | Value | P | P¹ |
| Experimental | 51 | 8.37 ± 3.26 | 0.885 | 12.37 ± 2.61 | 0.000 | 4.00 ± 3.24 | 0.000 | 0.000 |
| Control | 51 | 8.27 ± 3.56 | | 7.7 ± 2.95 | | −0.57 ± 3.59 | | 0.263 |

Note:
P represents the comparison between Experimental group and Control group.
P¹ represents the self-comparison before and after clinical trial.

### 6. The Effect of the Magnesium-L-Threonate and Phosphatidylserine-Based Formula on Free Recall of Pictures Memory Test

The average score of free recall of pictures memory test has no significant difference (P>0.05) between experimental group and control group before the clinical trial. Average score of free recall of pictures memory test in experimental group was higher than control group after the trial, with statistical significance (P<0.001).

In self-comparison, average score of experimental group after the trial was higher than before, with statistical significance (P<0.001). There is no significant difference of the average score of free recall of pictures memory test in placebo group before and after the trial (P>0.05).

TABLE 6
The effect of magnesium L-threonate and phosphatidylserine
on scores of free recall of pictures memory test.

| | | Before | | After | | Increased | | |
|---|---|---|---|---|---|---|---|---|
| Groups | Subjects | Trial | P | Trial | P | Value | P | P¹ |
| Experimental | 51 | 11.47 ± 5.04 | 0.938 | 16.65 ± 3.07 | 0.000 | 5.18 ± 3.27 | 0.000 | 0.000 |
| Control | 51 | 11.39 ± 5.11 | | 10.75 ± 4.46 | | −0.65 ± 2.52 | | 0.073 |

Note:
P represents the comparison between Experimental group and Control group.
P¹ represents the self-comparison before and after clinical trial.

### 7. The Effect of the Magnesium-L-Threonate and Phosphatidylserine-Based Formula on Scores of Recognition of Meaningless Figures Memory Test

The average score of recognition of meaningless figures memory test has no significant difference (P>0.05) between experimental group and control group before the clinical trial. Average score of recognition of meaningless figures memory test in experimental group was higher than control group after the trial, with statistical significance (P<0.001).

In self-comparison, average score of experimental group after the trial was higher than before, with statistical significance (P<0.001). There is no significant difference of the average score of recognition of meaningless figures memory test in placebo group before and after the trial (P>0.05).

TABLE 7
The effect of magnesium-L-threonate and phosphatidylserine on
scores of recognition of meaningless figures memory test.

| | | Before | | After | | Increased | | |
|---|---|---|---|---|---|---|---|---|
| Groups | Subjects | Trial | P | Trial | P | Value | P | P¹ |
| Experimental | 51 | 11.73 ± 4.61 | 0.597 | 17.88 ± 2.73 | 0.000 | 6.16 ± 3.99 | 0.000 | 0.000 |
| Control | 51 | 12.16 ± 3.54 | | 12.24 ± 1.54 | | 0.08 ± 2.76 | | 0.840 |

Note:
P represents the comparison between Experimental group and Control group.
P¹ represents the self-comparison before and after clinical trial.

8. The Effect of the Magnesium-L-Threonate and Phosphatidylserine-Based Formula on Scores of Recall of the Connection Between Portraits and their Characteristics Memory Test

The average score of recall of the connection between portraits and their characteristics memory test has no significant difference (P>0.05) between experimental group and control group before the clinical trial. Average score of recall of the connection between portraits and their characteristics memory test in experimental group was higher than control group after the trial, with statistical significance (P<0.001).

In self-comparison, average score of experimental group after the trial was higher than before, with statistical significance (P<0.001). In the placebo group, there was a small but significant decrease of the average score of recall of the connection between portraits and their characteristics memory test after the trial than before (P<0.001).

TABLE 8
The effect of magnesium L-threonate and phosphatidylserine on recall of
the connection between portraits and their characteristics memory test.

| | | Before | | After | | Increased | | |
|---|---|---|---|---|---|---|---|---|
| Groups | Subjects | Trial | P | Trial | P | Value | P | P¹ |
| Experimental | 51 | 9.82 ± 5.08 | 0.954 | 13.75 ± 2.42 | 0.000 | 3.92 ± 3.64 | 0.000 | 0.000 |
| Control | 51 | 9.76 ± 5.11 | | 9.20 ± 4.97 | | −0.57 ± 1.64 | | 0.017 |

Note:
P represents the comparison between Experimental group and Control group.
P¹ represents the self-comparison before and after clinical trial

### 9. The Effect of the Magnesium-L-Threonate and Phosphatidylserine-Based Formula on Memory Quotient (MQ) of Subjects

The MQ had no significant difference (P>0.05) between experimental group and control group before the clinical trial. MQ in experimental group was higher than control group after the trial, with statistical significance (P<0.001).

In self-comparison, MQ of experimental group after the trial was higher than before, with statistical significance (P<0.05). There is no significant difference of the MQ in placebo group before and after the trial (P>0.001) as shown in FIG. 2.

TABLE 9
The effect of magnesium-L-threonate and phosphatidylserine on MQ of subjects.

| | | Before | | After | | Increased | | |
|---|---|---|---|---|---|---|---|---|
| Groups | Subjects | Trial | P | Trial | P | Value | P | P¹ |
| Experimental | 51 | 60.31 ± 11.35 | 0.854 | 81.84 ± 7.18 | 0.000 | 21.53 ± 6.98 | 0.000 | 0.000 |
| Control | 51 | 60.75 ± 12.31 | | 61.73 ± 10.27 | | 0.98 ± 5.47 | | 0.206 |

Note:
P represents the comparison between Experimental group and Control group.
P¹ represents the self-comparison before and after clinical trial

### C. Conclusions

### 1. Safety Parameters

All subjects were tested normal for Chest X-ray, ECG, B-ultrasound before the study. Before and after taking the product magnesium-L-threonate, all participants have normal blood biochemical parameters including red blood cell counts, white blood cell counts, hemoglobin counts, platelet counts, serum total protein, albumin, alanine, aminotransferase, aspartate aminotransferase, urea, creatinine, cholesterol, triglycerides, and blood glucose levels.

Thus, it can be concluded that magnesium-L-threonate is safe for humans. There were no side effects observed during the study.

### 2. Functional Parameters

i. Between the Groups

After sample treatment, the MQ and the average scores of the five subcategory memory tests, directed memory, paired-association learning, free recall of pictures, recognition of meaningless figures, recall of the connection between portraits and their characteristics in experimental group were higher than control group, with statistical significance (P<0.001).

i. In Group Comparison (Before and After Comparison)

The experimental group has higher MQ after treatment, and the average score of the five subcategory memory tests, directed memory, paired-association learning, free recall of pictures, recognition of meaningless figures, recall of the connection between portraits and their characteristics were also higher after the treatment than before, all with statistical significance (P<0.001).

In the placebo group, there was a small but significant decrease of the average score of recall of the connection between portraits and their characteristics memory test after the trial than before (P<0.001). All the rest of indexes in the placebo group has no significant difference (P>0.05) before and after.

### 3. Drop-Out Rate

There were total 109 participants enrolled in the study, including 54 subjects for experimental group and 55 subjects for control group. 102 subjects finished this trial, 54 subjects for experimental group and 55 subjects for control group. 3 subjects in experimental group and 4 subjects in control group failed to follow up due to loss of contact. Total drop-out rate was 6.42%.

### D. Discussion

Magnesium and ascorbic acid, the raw materials used to obtain magnesium-L-threonate (Magtein®), have a long history of use since they are naturally present in foods. Magnesium is one of the most abundant minerals in the human body and of great importance for proper body functioning. It is present in grains, cereals, and dark leaves such as spinach and cabbage.

Ascorbic acid participates in several biochemical functions, helps the immune system, acts as an antioxidant, and is present in citrus fruits such as orange and acerola and vegetables such as peppers, spinach, and arugula.

The brain naturally undergoes structural and functional changes even in the absence of neurodegenerative diseases. Today, it is known that from the age of 25, the brain volume begins to decrease. This type of alteration affects the cognitive health and leads to the loss of memory, tension, anxiety, dementia, Alzheimer's disease, phobias, and sleep disturbance among others conditions.

The world aging population is growing rapidly, posing a public health challenge. Nowadays, the most common problems affecting the elderly are related to memory and cognition, such as Alzheimer's disease, which is the most common form of dementia among this population, characterized by an accelerated process of cognitive degeneration. In this context, the effects of magnesium-L-threonate (Magtein®) in combination with a neurotransmitter was investigated in this trial. Before the trial, two groups were of the same level of memory quotient (MQ) and five subcategories of memory tests. After treatment, the MQ and average scores of the five subcategories of memory tests in the magnesium-L-threonate and neurotransmitter-based formula group were higher than the control group, with statistical significance (P<0.001). In addition, comparing before and after within the same group, the magnesium-L-threonate and neurotransmitter-based formula group had a significantly higher MQ and average score of the five subcategory memory tests (P<0.001) while the placebo group had no change, except a small but significant decrease of the average score of recall of the connection between portraits and their characteristics memory test (P<0.05). This data supports the strong benefits of the magnesium-L-threonate (Magtein®) and neurotransmitter-based formula on improving memory and cognition in this group of the adult subjects from among the healthy Chinese population.

### E. Comparison of Results of Examples 1 and 2

According to the obtained data of Example 1, the magnesium-L-threonate-based formula has demonstrated significant benefits to improving memory and cognition.

When the results of Example 1 are compared to the results of Example 2, the improvement of cognitive function by the magnesium-L-threonate and phosphatidylserine-based formula (Magtein®PS) is significantly higher than that of the Magtein®. As shown in FIG. 3, the improvement in cognitive function for the Magtein®PS is about twice as great as for Magtein® (p<0.001). Thus, the magnesium-L-threonate and phosphatidylserine-based formula has a synergistic effect on cognitive brain health that far surpasses the effect on cognitive brain health of magnesium-L-threonate alone.

In Example 3, below, a total of 60 male rats were divided into 4 groups with 15 in each. The animals were randomized based on stratified body weight and treated with vehicle or test compounds for a period of 21 days. On day 16th, Morris Water Maze (MWM) training was initiated with two trainings per day for a period of 5 days (day 16-20), on day 21, probe test was carried out for the cognitive assessment using MWM tests. MWM data was analyzed by ANYMAZE software. Administration of Magnesium-L-threonate (Magtein®) produced significant increase in distance travelled in the target quadrant, when compared to vehicle. A similar observation was recorded with phosphatidylserine (PS). Interestingly, a combination treatment that included both Magtein and PS produced a synergistic effect in the memory retrieval compared to individual drug treatment or vehicle group.

### Example 3

### a. Materials and Methods

### 1. Animal Welfare

This study was carried out following all ethical practices laid down in the guidelines for animal care at Central for Experimental Pharmacology and Toxicology (CPT) JSSAHER, Mysuru, a CPCSEA (Registration number 261/PO/ReBi/S/2000/CPCSEA).

This study has been approved by the Institutional Animals Ethics Committee (IAEC) of the test facility and the approval No. JSSAHER/CPT/IAEC/055/2020.

TABLE 10

| | |
|---|---|
| Animal species | Male Wistar Rats |
| Justification for | Rat is the standard laboratory rodent |
| selection of species | species used for cognition models |
| Source | Charles River Laboratories |
| | Hylasco Biotechnology (India) Pvt. Ltd. |
| | CPCSEA 1808/PO/RcBt/S/15/CPCSEA |
| | Hyderabad-500 078 |
| Number of | 15 male rat/ group |
| animals/group | |
| Age of animals | 7-8 weeks |
| Randomisation | Animals were selected and/or |
| | grouped by computerized excel sheets. |
| Treatment | Through drinking water |
| | for 21 consecutive days |

### 2. Study Design

The selected male rats were assigned to vehicle control and treatment groups that were administered either magnesium-L-threonate (MgT), phosphatidylserine (PS) or a combination of magnesium-L-threonate and phosphatidylserine (MgT+PS) as shown in Table 11:

TABLE 11

| Group | Treatment | Dose | Route of | No. of |
|---|---|---|---|---|
| No. | group | (mg/kg bw/day) | administration | rats |
| G1 | Vehicle control | 0 | Through | 15 |
| G2 | MgT | 435 MgT | Drinking | 15 |
| G3 | PS | 60 PS | Water | 15 |
| G4 | MgT + PS | 435 MgT + 60 PS | | 15 |

### 3. Test Method: Morris Maze Test (MWM)

MWM was used to evaluate the spatial learning and memory in rodents. The ability of the rat to identify the hidden platform with the help of cues in the maze is assessed as the learning and memory function.

### 4. Apparatus

The maze consists of a circular tank 125 cm diameter, 36 cm height placed on top of a stand, 20 cm above the floor. The maze is equally divided into four quadrants Q1, Q2, Q3, Q4 and create an imaginary ‘plus’ (+) sign. Each quadrant is designated as SE (South East), SW (South West), NE (North East) and NW (North West). A platform with 10 cm2 diameter is placed in the Q3, which is kept permanently during training phase. The maze was filled with water to a level 1 cm above the platform. Water was made opaque/blurred with the help of a nontoxic material and maintained at a 28-35° C. in order to prevent hypothermia.

### 5. Morris Maze Steps

i. Training Phase

The rat was gently placed in a fixed quadrant (the introduction position was the same throughout training and probe trails) with the head of the animal facing towards the wall of the maze. The rat was allowed to locate the hidden platform in the third quadrant. Rats which were unable to find the hidden platform were guided to identify the platform. Once the animal climbed the platform, they were allowed to explore for 15 seconds. Then the rat was taken out of the maze wiped, dried and then returned into the cage. The escape latency (EL)—period it took for the animal to reach the safe platform, path length, and the time for the rat to enter into the target were assessed using a video tracking software-Any Maze, USA. Animals were trained twice a day for five days (day 16 to day 20).

### 6. Statistical Analysis

Data was presented as Mean±SD/SEM. Statistical analysis was performed using GraphPad Prism software and other statistical software.

EL, the number of entries in the target quadrant, and the path length were captured using a 12 MP camera and the data was analyzed using video Any Maze, USA software.

### B. Results

No treatment related mortality was observed.

There was no treatment related clinical signs observed.

### 1. Cognitive Functions as Measured Through Morris Maze Test

i. Time Spent in Target Quadrant:

MgT and PS treated groups showed significant increase in the time spent in the target quadrant when compared to the vehicle treated group.

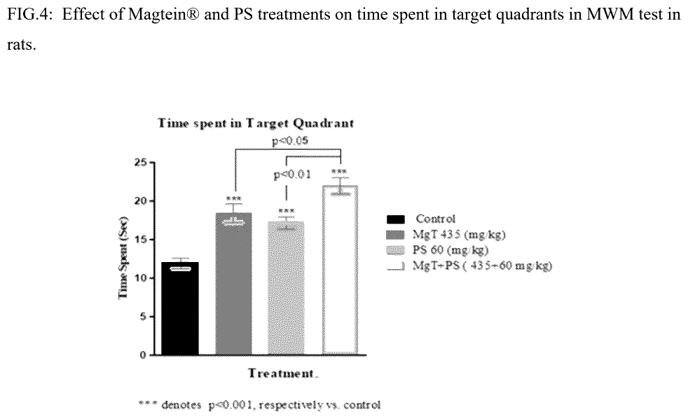

The combination groups have shown synergistic increase in the time spent in target quadrant, as compared to the control (P<0.001), as well as MgT (p<0.05) and PS alone (p<0.01) as shown in FIG. 4.

### C. Conclusion

Based on the MWM test results, it is evident that co-administration of MgT with PS produces a synergistic nootropic effect in the rats in the test conditions. This conclusion is derived based on in-life observation and MWM test data.

We claim:

1. A composition comprising magnesium-L-threonate and about 200 mg of phosphatidylserine.

2. The composition of claim 1, wherein the phosphatidylserine is derived from soy.

3. The composition of claim 1, wherein the phosphatidylserine is derived from at least one of the group consisting of sunflower, fish, krill, and bovine.

4. The composition of claim 1, further comprising at least one of vitamin C, vitamin D3, vitamin B6, and vitamin B12.

5. A method of improving at least one of memory and cognition in a subject, the method comprising:
identifying a subject having a deficiency in at least one of memory and cognition; and
administering a therapeutically effective amount of a composition comprising magnesium-L-threonate and a daily dosage of about 200 mg of phosphatidylserine.

6. The method of claim 5, wherein the phosphatidylserine is derived from soy.

7. The method of claim 5, wherein the phosphatidylserine is derived from at least one of the group consisting of sunflower, fish, krill, and bovine.

8. The method of claim 5, further comprising at least one of vitamin C, vitamin D3, vitamin B6, and vitamin B12.
